(19) United States
(12) Patent Application Publication
(10) Pub. No.: US 2006/0241399 A1
(43) Pub. Date: Oct. 26, 2006

(54) Multiplex system for the detection of surgical implements within the wound cavity

(76) Inventor: Carl E. Fabian, Miami, FL (US)

Correspondence Address: ROBERT M. SCHWARTZ, P.A., P.O. BOX 221470, HOLLYWOOD, FL, 33022, US

(21) Appl. No.: 11/323,379

(22) Filed: Dec. 30, 2005

Related U.S. Application Data
(63) Continuation-in-part of application No. 11/054,844, filed on Feb. 10, 2005.

(51) Int. Cl. A61B 5/05

(52) U.S. Cl. 600/424

(57) ABSTRACT

The present invention provides a multiplex system for identifying the presence of surgical sponges, metal instruments, and other implements in a surgical wound by employing a plurality of discrete sensing systems. At least two modalities of operation are chosen from a plurality of detection modalities, which can include: 1) magnetomechanically resonant marker tags; 2) “smart markers” or RFID markers; and 3) a system designed to detect metallic objects solely based upon their metal content, without need for a separate, affixed marker. The selected modes of operation can operate sequentially or simultaneously. Consequently, the use of such a multiplex system eliminates the possibility that surgical implements, non-metallic or metallic, will be left behind within a surgical cavity.

### PRIORITY

The present application claims priority from co-pending U.S. Utility patent application Ser. No. 11/054,844, Filed on Feb. 10, 2005 and entitled MULTI-MODAL DETECTION OF SURGICAL SPONGES AND IMPLEMENTS.

The present application is related to U.S. patent application Ser. No. 11/055,348, filed on Feb. 10, 2005 and entitled SURGICAL IMPLEMENT DETECTOR UTILIZING A RADIOFREQUENCY IDENTIFICATION MARKER, and a continuation-in-part application thereto, U.S. patent application Ser. No. (Not Yet Assigned), filed on Dec. 9, 2005, and entitled SURGICAL IMPLEMENT DETECTOR.

### BACKGROUND OF THE INVENTION

1. Field of the Invention

The present invention relates to a system comprised of multiple detecting modalities, herein called a “multiplex system”, for detecting non-metallic and metallic surgical implements, and more particularly to a system using at least two different modalities operative for the detection of surgical implements made of metal and/or bearing detectable tags.

2. Description of the Related Art

Many patents disclose methods for detection of surgical implements at the completion of surgery, prior to wound closure. Such detection methods incorporate x-ray opaque markers within surgical implements and effect detection using postoperative x-ray of the patient or of the discarded sponges. Also disclosed as being suitable for detection of surgical implements are methods involving use of resonant tags made from magnetomechanical elements, capacitors, LRC oscillatory circuits or smart markers.

U.S. Pat. No. 3,097,649 to Gray discloses a method and application for detecting a surgical sponge. The sponge carries a radioactive material such as uranium oxide sewn therein. Radiation emitted from the sponge is detected by a Geiger counter, prior to surgical wound closure. No disclosure is contained therein concerning a method for detection of retained metallic implements.

U.S. Pat. No. 3,508,551 to Walters et al. discloses dressings and production thereof. An x-ray detectable opaque filament is incorporated within the dressing. For use of this device, the patient must be transported from the operating room to an x-ray room. The process is cumbersome and exposes the patient to unnecessary radiation. Detection of a retained dressing tends to be limited due to the small diameter of the x-ray opaque filament. The dressing can be overlooked when orientation of the filament is directly in-line with a bone.

U.S. Pat. No. 3,587,583 to Greenberg discloses a surgical sponge with magnetizable means. The sponge has a flexible thread with magnetizable particles. A plurality of magnetizable barium ferrite particles are embedded in a plastic material, such as nylon, forming a flexible magnetizable thread. The surgical instruments used may also be provided with a small amount of magnetizable material. A surgical cavity is probed using a magnetic detection means such as a magnetodiode. The Greenberg disclosure does not state how the surgical instruments could be made to have magnetizable material. The size of threads is too small to be detected unless the probe is also inserted into the surgical cavity, which procedure would likely present issues involving sterility and tissue damage.

U.S. Pat. No. 3,686,564 to Mallick, Jr. et al. discloses a multiple frequency magnetic field technique for differentiating between classes of metal objects. Low and high frequency oscillators are mixed using multiple frequency excitation. The magnetic field generated is examined by observing the voltage current vectorial relationship. When metal is present in the incident electromagnetic field, the vectorial relationship is changed according to the size, shape and type of a metallic object. The system is primarily designed to detect large metallic objects, such as guns in an airport, and is a walk-through arrangement. No disclosure is set forth concerning detection of small metallic objects such as a surgical implement in a surgical incision.

U.S. Pat. No. 3,698,393 to Stone (hereinafter the '393 patent) discloses a surgical pad. A radiopaque plastic ring is attached to the pad. After a surgical procedure is complete, the patient is radiographed to determine whether a sponge having an attached plastic ring has been retained within the surgical cavity. The system requires that the patient be transported to an X-ray facility; it exposes the patient to unnecessary radiation.

U.S. Pat. No. 3,834,390 to Hirsch (hereinafter the '390 patent) discloses a neurological sponge. The sponge has a double layer comprising a highly absorbent inner layer wrapped by a porous outer layer. An x-ray detectable BaSO4 material wrapped in plastic is placed between the two layers. The x-ray detectable material absorbs x-rays, indicating the presence of the sponge within a surgical cavity. Prior to closure of the surgical incision, the patient must be transported to an x-ray facility, at which location the patient is exposed to unnecessary x-ray radiation.

U.S. Pat. No. 3,964,041 to Hinds discloses an article detection system and method. Articles such as container ends are sensed and detected to provide count and/or control outputs representative of the number of such articles detected. A frequency sensitive detector circuit generates a fixed frequency signal, which is utilized to modulate the output from a signal generating transducer or signal source. The modulated output from the source impinges on an article being sensed, which reflects or interrupts this signal. The reflection or interruption is sensed by a suitable transducer or sensor. A feedback signal generated by the transducer or sensor is fed back to the frequency sensitive detector that generated the original fixed frequency signal. The detected signal is the same as the frequency of the original modulating signal. One of these output signals is indicative of the detection of an article, and is applied to suitable counting and/or control circuitry that provides the desired count and/or control outputs. A feedback signal indicates the presence of an article and provides an accurate count of articles, such as can ends. The article detection system and method disclosed by the Hinds patent does not detect metallic objects or non-metallic sponges inadvertently retained within a surgical incision.

U.S. Pat. Nos. 4,114,601 and 4,193,405 to Abels disclose a medical and surgical implement detection system. Surgical implements, surgical instruments, surgical sponges, surgical implantable devices and indwelling therapeutic devices and materials are detected within the human body or other area of interest by incorporating or adding a radiofrequency transponder. This is a microwave system that mixes two fundamental microwaves having 4.5-5 GHZ frequencies, and relies on a non-linear transponder to produce higher order product frequencies. The transponder may be a thin film of a ferrite material exhibiting gyro-magnetic resonance at selected frequencies. Alternately, the transponder may be a solid-state device containing diodes and field effect transistors. This non-linear transponder signal is received by a receiving antenna and is filtered to remove all fundamental microwave frequencies. Each of the higher order microwave frequencies generated by the transponder is easily absorbed by the human body. Consequently, most of the signal is lost before any non-linear transponder can be detected. Additionally, the absorption of the signal results in undesired heating of body tissue. Further, the gyro-magnetic effect produces only a weak signal.

U.S. Pat. No. 4,658,818 to Miller, Jr., et al., discloses an apparatus for tagging and detecting surgical implements. A miniature battery-powered oscillator is attached to each surgical implement and activated prior to its initial use. The output of each oscillator is in the form of a low powered pulse of 1-10 MHZ frequency and is coupled to the body's fluids and tissue. After the surgery is completed, but prior to suturing, a detection system is used to sense for any pulses generated by the oscillator within the body. The surgical implement detection system disclosed by the '818 patent is not passive. It requires a miniature battery, which remains in the “on” condition from the beginning of the operation. If a sponge is left behind, the microwave radiation is detected. When the operation is complete, the battery might have discharged, in which case the sponge would not be detected. In addition, the system disclosed by the '818 patent does not detect metallic objects and, as previously noted, use of microwave frequencies can cause undesired heating of body tissue.

U.S. Pat. No. 5,057,095 to Fabian discloses a surgical implement detector utilizing a resonant marker for use in human or animal tissue. The marker is set into resonance by the interrogating field and the resonance frequency signal emitted by the marker is detected by a separate detection circuit adjacent to the interrogating circuit. The marker resonates due to magnetostriction properties of an amorphous ribbon or piezoelectric device or a tuned LRC circuit. The marker is a single function device and the system only detects surgical implements to which a marker has been secured. Its size of approximately 2 inches makes it not optimal for attachment to smaller implements or gauze sponges and its ability to survive heat sterilization makes it suboptimal for use on metallic implements. Furthermore, even when such a marker is secured to a metallic implement, metal in close proximity may cause distortion of the signal, so-called ‘shielding’, reducing the reliability (i.e., the range) of detection.

U.S. Pat. No. 5,099,845 to Besz et al. discloses a medical instrument location means. A powered radiating element is attached to a device appointed for insertion into the body. The location of the radiating element within the body is assessed by moving a handheld receiving unit over the external surface of the body to obtain a maximum radiation value, thereby pointing the receiving sensor directly above the radiating element. Next, the intensity of the radiation energy is assessed to determine how deep the radiating element is located from the surface of the body. The radiating element requires power to operate and, therefore, does not detect unpowered metallic objects or sponges even if they contain a passive tag. Powered tags are also subject to considerations of battery life. Should such a tag exceed its battery life during an operation, the tag would become undetectable.

U.S. Pat. No. 5,541,604 to Meier discloses transponders, interrogators, systems and methods for elimination of interrogator synchronization requirement. A Radiofrequency Identification (RFID) system has an interrogator and a transponder. The interrogator has a first tuned circuit of a powering frequency for sending a powering burst to a transponder. A filter/demodulator receives a wireless, modulated RF response from the transponder. The interrogator also has a second tuned circuit in electrical communication with a modulator. The second tuned circuit has a selected bandwidth about a communication frequency. The selected bandwidth does not substantially overlap the powering frequency; but encompasses the bandwidth of the modulated carrier of the RF response. The carrier is modulated using pulse width modulation (PWM), pulse position modulation (PPM), frequency-shift keying modulation (FSK), or other modulation method. The interrogator also has a controller in electrical communication with the filter/demodulator and the tuned circuits for enabling the first tuned circuit to send the powering burst during a first time period and of enabling the modulator in electrical communication with the second tuned circuit to receive the RF response during a second time period. The transponder has a tuned circuit, a tuning circuit in electrical communication with the tuned circuit for modifying the frequency characteristics of the tuned circuit such that it is can be tuned during the powering burst to the powering frequency, and be tuned during the RF response to the communication frequency. The transponder also includes a demodulator in electrical communication with the tuned circuit for receiving the RF interrogation therefrom and for demodulating data from the RF interrogation. This current generation RFID device sends a preset code to the interrogator and is powered entirely by the power burst signal provided during the first time period. It is capable of transmitting the code at a high rate to the interrogator.

U.S. Pat. Nos. 5,650,596 and 5,923,001 to Morris, et al. disclose an automatic surgical sponge counter and blood loss determination system. Each sponge carries an RF tag which is read by a sensor located in proximity with the opening of a soiled sponge-receiving container provided with a disposal bag. The disposal bag is weighed and its dry weight compared based on the ID of the sponge tag. The weight of blood and other body fluids is determined by subtraction. A display is used to provide information about sponges in the container, and the weight of blood and body fluids dispensed within the container. The system of the '596 patent does not detect sponges retained within a patient during an operation; it only counts surgical implements when they are disposed within the container. The '001 patent discloses a handheld RF reader to be passed over the surgical site by a surgeon to detect the presence of surgical sponges in the body cavity at the time of closure during a surgical procedure. The '001 patent states that the handheld RF reader will then identify any sponges which may have been inadvertently left in the wound, thus preventing the retention of sponges inside of the patient. This system is intended to detect only tagged sponges. It does not detect metal instruments or other implements.

U.S. Pat. No. 5,944,023 to Johnson et al. discloses systems and methods for determining the location of an implanted device including a magnet. The tip of the body-inserted implanted device includes a generating mechanism which may be a permanent magnet or a permanent direct current magnet with a self-induced magnetic field. The location of the magnet is detected outside the patient by a mat, which incorporates a multitude of magnetic field sensors. The magnet positional information is displayed on a video screen. This system disclosed by Johnson does not locate surgical instruments or sponges within a surgical cavity.

U.S. Pat. Nos. 6,009,878 and 6,305,381 to Weijand et al. disclose a system for locating an implantable medical device. This system has an implanted coil, which transmits electromagnetic radiation and is picked up by an electromagnetic energy receiving device with three symmetrically oriented coils external to the patient. When the energies received by these three coils are equal, the receiving device is directly above the implanted coil, and the drug reservoir in the implant may be filled. This system does not detect medical instruments or sponges accidentally retained by the surgical wound of a patient during an operation.

U.S. Pat. No. 6,057,756 to Engellenner discloses electronic locating systems. Coded tags are interrogated at various locations in the intended path of a transportation vehicle. The presence of a vehicle in a specific location is determined and relayed to a central controller. The '756 patent discloses a system for managing and tracking a transportation process. No disclosure is contained within the '756 patent concerning detection of metallic objects or sponges accidentally left behind in a surgical incision after completion of surgery.

U.S. Pat. No. 6,076,007 to England et al. discloses a portable unit for detecting the presence of surgical devices, and their location. A high permeability, low coercivity, wire or strip tag is implanted with a surgical device. The tag is interrogated by a search coil energized by a high frequency AC field with a DC or low frequency bias filed. Phase information is used to detect the directionality of the tag location. The detection system is based on flying null technology. It is a single functionality detection system, and does not detect metallic objects that are not incorporated with a tag. Metallic objects adjacent to the tag may distort phase information providing an unreliable indication.

U.S. Pat. No. 6,615,155 to Gilboa discloses object tracking using a single sensor or a pair of sensors. The three dimensional movement of a moving object is tracked by measuring one or more vector fields assisted by theoretical computations. The system does not track or detect stationary objects such as a sponge or metallic object accidentally included in a surgical incision.

U.S. Pat. No. 6,026,818 to Blair, et al discloses a tag and detection device. An inexpensive tag has the form of a ferrite bead with a coil that resonates at a particular frequency, or a flexible thread composed of a single loop wire and capacitor element. The detection device locates the tag by pulsed emission of a wide-band transmission signal. The tag resonates with a radiated signal, in response to the wide band transmission, at its own single non-predetermined frequency, within the wide band range. This system does not detect untagged metallic surgical implements.

U.S. Pat. No. 6,424,262 and US Patent Application publication No. 20040201479 to Garber, et al. disclose applications for radiofrequency identification systems. An RFID target cooperates with a magnetic security element and a bar code reader to check out and manage library materials such as reference books, periodicals, and magnetic and optical media. No disclosure is contained with the '262 patent and '479 patent Application concerning detection of sponges or surgical pads in a surgical wound.

US Patent Application Publication No. 20030066537 to Fabian, et al. discloses surgical implement detection system. Surgical implements used during an operating procedure are detected in human tissue. Markers attached to the surgical implements change their impedance at a preselected frequency in the presence of an electromagnetic field. The system uses a magnetomechanical element which vibrates at a preselected frequency when excited, and this preselected frequency is detected, indicating the presence of a surgical implement to which the magnetomechanical marker element is attached. Such a system does not detect untagged metallic surgical implements.

US Patent Application Publication No. 20030176785 to Buckman et al., discloses a method and apparatus for emergency patient tracking. This tracking system attaches a coding device to a patient and is tracked. In fact, the coded device utilized is associated with each patient in such a way that the device cannot be removed or disassociated from the patient without a concerted effort. Such a system does not detect accidentally included sponges or metallic objects in a surgical incision.

PCT Patent Application No. WO 98/30166 and European Patent Specification 1 232 730 A1 to Fabian et al. disclose a surgical implement detector utilizing a smart marker. The marker is coded and the code is transmitted through an antenna to a central microprocessor, which verifies the code. Each marker has to be individually coded and inserted into a sponge surgical pad, etc. The system does not detect untagged metallic objects left behind within a surgical incision.

PCT Patent Application No. WO 03/032009 to Fabian et al. discloses a surgical implement detection system. A marker attached to the surgical implement changes its impedance at a preselected frequency in the presence of an electromagnetic interrogating field. The interrogating electromagnetic field has a preselected frequency, preferably modulated as a series of pulses and the marker, a magnetomechanical element, attached to the surgical implement resonates at a preselected frequency in response to the field. The detector detects a ring-down signal of the marker between the pulses. This system does not detect metallic objects that do not have a marker attached. Additionally, if a tag is attached to a metallic implement, the close proximity of metal may result in distortions of the signal, or “shielding” weakening the signal received, and possibly allowing it to escape detection.

PCT Patent Application No. 03/048810 and US Patent Application Publication No. 20030105394 to Fabian et al. disclose a portable surgical implement detector. The portable detector interrogates a marker that is attached with a surgical implement which signals a preselected frequency. This system does not detect metallic objects that do not have a marker attached. Besides, as noted in the foregoing paragraph, a metallic surgical implement may shield a marker, causing weakening of the preselected frequency signal.

US Patent Application Publication No. 20030192722 to Ballard discloses a system and method of tracking surgical sponges. The sponges have a radiopaque object embedded therein which is visible when the sponge is x-rayed. All sponges removed from the surgical wound are placed into a sponge container which is provided with an internal device to x-ray and identify the sponges after use. Sponges are x-rayed only once, after use, as they are placed in the container for disposal. Sponges are not x-rayed before being inserted into the wound, and cannot, by the device of Ballard, be x-rayed while in the patient. Therefore, the process cannot actively detect whether a sponge has been accidentally left behind in a surgical wound nor does it provide a way of knowing that all the sponges have been removed, or whether any have been left behind in the patient wound.

US Patent Application Publication No. 20040129279 to Fabian, et al. discloses a miniature magnetomechanical tag for detecting surgical sponges and implements. This tag is a magnetomechanical device, and is excited by the interrogating magnetic field. The interrogating field is switched off and the ring down of the resonant target is detected. This system does not provide means for identifying an individual sponge or surgical pad.

US Patent Application No. 20040250819 to Blair, et al discloses an apparatus and method for detecting objects using tags and wideband detection devices. An apparatus and method for the detection of objects in the work area such as surgical sites, including a detection tag affixed to objects such as used during surgery, is disclosed. The apparatus and method feature interrogates with a transmitter emitting a pulsed, wideband signal. This signal prompts the tag element to provide a return signal, which is received and analyzed. The device features an antenna portion containing a single or a plural ring-shaped antenna. Also, the pulsed wideband interrogation signal may be pulse-width modulated or voltage-modulated. The pulsed signals trigger a continuing response signal from the tag in its response frequency range, which increases in intensity to the point where it becomes differentiable from background noise and is detected within the wideband range by the signal detector as an indication of the presence of the tag. The tag is excited by a wide-band pulsed interrogation signal, which builds up the output of the tag and can be detected over ambient electronic noise. The tag signal has a predetermined frequency. It is a sinusoidal wave, and does not carry digital information identifying the sponges and surgical pads used.

US Patent Application Publication No. 20050003757 to Anderson discloses an electromagnetic tracking system and method using a single-coil transmitter. The system includes a single coil transmitter emitting a signal, a receiver receives a signal from the single coil transmitter. Electronics process the signal received by the receiver. The electronics determine a position of the single coil transmitter. The transmitter may be a wireless or wired transmitter. The receiver may be a printed circuit board. The electronics may determine position, orientation, and/or gain of the transmitter. The single coil transmitter is a powered device and may be wired or wireless. It is not a passive device that can be incorporated in a sponge or surgical pad due to the requirement for a reliable power source. Powered tags are also subject to considerations of battery life. Should such a tag exceed its battery life during operation, the tag would become undetectable.

PCT Patent Application No. WO 98/30166 to Fabian et al. discloses a surgical implement detector utilizing a smart marker. The surgical implement is appointed for disposition within human or animal tissue and is caused to become electronically identifiable by affixing thereto a smart marker, which is an unpowered integrated circuit with an EEPROM memory that carries a code. When a smart marker is sufficiently close to the reader antenna, a voltage is generated within the marker antenna that charges the capacitor and powers the integrated circuit. A switch is opened and closed to transmit the stored code in the EEPROM memory, providing identification and recognition of a smart target attached to a surgical sponge. The marker antenna operates at a frequency of near 125 KHz. Frequency of information transfer to the reader is very slow due to the switching on and off action. In addition, the smart marker is not encapsulated and is subject to damage by blood and other saline fluids. Further, if applied to metallic implements, the smart marker may be subject to “shielding” and weakening of the signal, as described above, possibly making detection of tagged metallic implements unreliable.

There remains a need in the art for a highly reliable surgical implement system that detects both non-metallic surgical implements including sponges, laparotomy pad and gauze and metallic surgical instruments, so that none of these surgical implements are left behind in a surgical incision after wound closure. The procedure for detection of accidentally included surgical implements must be exceedingly reliable. Apparatus employed to practice this procedure should be easy to operate so that the patients can be routinely examined without complex set-up and teardown steps, thereby enabling risk of infection and rejection reactions to be minimized.

Ideally markers or tags secured to surgical implements would be tiny in size, inexpensive, universally detectable through air or tissue at a reasonable distance, unaffected by proximity to metal, biologically inert and harmless to the patient and personnel, robust enough to survive any means of sterilization, and able to carry coded data to provide a record of all items so tagged.

In view of the limitations inherent in each detection system noted above, there presently exists no single modality capable of reliably detecting all of the implements used in modern surgery. None of the references, discussed above, disclose using more than one modality. What is needed is a combination of two or more modalities, each with its particular capabilities, operating together to ensure that no items are inadvertently overlooked and left behind in the wound.

### SUMMARY OF THE INVENTION

The present invention provides a multiplex system employing a plurality of discrete sensing systems for identifying the presence of surgical implements in a surgical wound. As used herein the term “implements” includes metal instruments, surgical sponges, gauzes, and other items used during a surgical operation.

Pursuant to the present invention, a highly reliable surgical implement detection system is provided which utilizes together, at least two different modalities of detecting surgical implements including sponges left in a surgical incision or body cavity.

One particular embodiment of the instant invention selects at least two modalities from the group of:

1) magnetomechanically resonant marker tags; 2) “smart markers” or RFID markers; and 3) a system designed to detect metallic objects solely based upon their metal content, without need for a separate, affixed marker. Generally stated, the invention employs the use of a combination of discrete systems to identify surgical implements. Consequently, the use of such a multiplex system eliminates the possibility that surgical implements, non-metallic or metallic, will be left behind within a surgical cavity.

Other features which are considered as characteristic for the invention are set forth in the appended claims.

Although the invention is illustrated and described herein as embodied in a Multiplex System For The Detection Of Surgical Sponges And Implements Within The Wound Cavity, it is nevertheless not intended to be limited to the details shown, since various modifications and structural changes may be made therein without departing from the spirit of the invention and within the scope and range of equivalents of the claims.

The construction of the invention, however, together with additional objects and advantages thereof will be best understood from the following description of the specific embodiment when read in connection with the accompanying drawings.

### BRIEF DESCRIPTION OF THE DRAWINGS

The invention will be more fully understood and further advantages will become apparent when reference is had to the following detailed description of the preferred embodiments of the invention and the accompanying drawings, in which:

FIG. 1 is a schematic diagram showing an operation in progress in which a multiplex detection system is in use;

FIG. 2A is a schematic diagram showing a magnetomechanically resonant marker;

FIG. 2B is a schematic diagram showing details of an RFID marker;

FIG. 3 is a diagrammatic representation of a surgical sponge provided with a magnetomechanically resonant marker tag;

FIG. 4 is a diagrammatic representation of a gauze pad provided with an RFID tag;

FIG. 5A is a hemostat provided with an RFID target which is directly secured to the hemostat;

FIG. 5B is a hemostat provided with an RFID target which is secured to the hemostat by a nonmetallic spacer;

FIG. 6A is a plan view of a metallic surgical instrument detectable by a metal-detecting mode of one embodiment of the instant application, without the need for a tag or marker; and

FIG. 6B is a plan view of another metallic surgical instrument detectable by a metal-detecting mode of one embodiment of the instant application, without the need for a tag or marker.

### DESCRIPTION OF THE PREFERRED EMBODIMENTS

The invention, called herein a multiplex system, comprises a plurality of discrete sensing systems to detect the presence of surgical implements, including surgical sponges, metal instruments, and other implements within the surgical wound, thereby preventing their inadvertent retention as the wound is closed. The term “implements” as used herein, shall encompass all devices that may be left in surgical wound, including, but not limited to, surgical sponges, laparotomy pads, gauze, rubber tubes, and metal instruments, such as clamps, retractors, hemostats and scalpels.

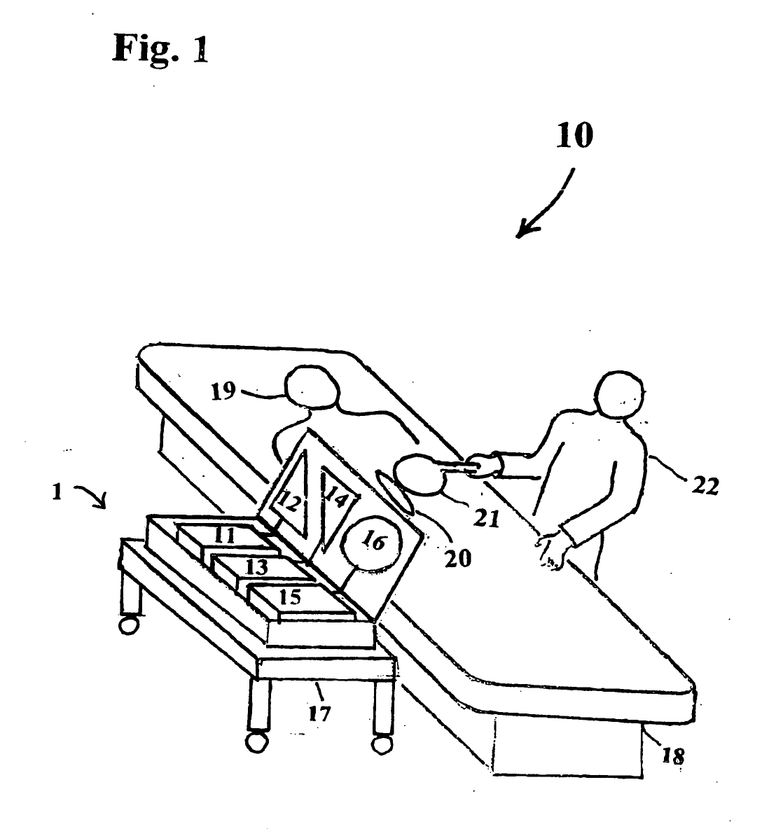

Referring now to FIG. 1, there is shown a schematic diagram showing an operation 10 in which a multiplex detection system 1 comprising three separate detecting systems is deployed. Note that although, for purposes of example, all three possible modes of detection are shown being deployed in FIG. 1, the instant invention is not meant to be so limiting. Rather, it is possible that, for purposes of the instant invention, as few as two of the three possible modalities may be used to detect sponges and other implements in a surgical incision or wound cavity. Additionally, if only two modalities are selected for use, any two of the modalities, disclosed herebelow, may be selected, based on the specific criteria of the user. Further, although only three modalities are illustrated in FIG. 1, the present invention can utilize more than three modalities, if desired. Further, although three very particular modalities are shown in the example of FIG. 1, these modalities are examples, and, as such, other detection modalities not shown in FIG. 1 may be used.

Referring back to FIG. 1, there is shown a first system 11, second system 13, and third system 15. In the present embodiment, the systems 11, 13 and 15 are incorporated on a rollaway cart 17, so as to be brought into close proximity to an operating table 18 supporting a patient 19 having an incision 20. The rolling cart 17, as shown in FIG. 1, can be brought close to the patient during the surgical procedure but prior to closing of the incision. Antennae associated with each system may be employed on the cart 17, or by a handheld antenna 21. Alternately, a multiplex detection system containing two or more of the systems 11, 13 and 15 can be brought into proximity with the patient in some other way, such as by being built into the table 18, or into some other permanent piece of equipment in the operating room. Further, the systems 11, 13 and 15 or other modalities can be made small enough and light enough to be personally transported, such as using a backpack or other portable mechanism.

The systems 11, 13 and 15 are provided so as to use at least two, and if desired, all three (or more, if desired), modalities of detection for sponges and other implements that may be placed in the surgical wound 20. Of the three possible modes of detection used, the systems 11 and 13 have been chosen in the particular embodiment to be modalities that utilize markers secured to various sponges and/or other surgical implements, which markers are capable of being detected electronically. The system 15 has been selected to provide a possible third detection modality, wherein the ferrous content of a metallic surgical implement is detected by the system 15, without the need for affixing an additional marker or tag. All three possible detection modalities will be described below.

Markers:

As described above, the systems 11 and 13 of FIG. 1 use electronic markers, or tags, affixed to, or otherwise in communication with, a surgical implement (such as a sponge, gauze, clamp, retractor, hemostat, etc.) Such an electronic marker would, ideally, be:
very small;
inexpensive;
instantly and universally detectable through body tissue at a range of up to 75 cms;
biologically inert;
robust enough in construction to withstand the usual methods of sterilization;
unaffected by proximity to metal; and
able to store coded information.

At present no such marker exists but this field continues to draw intense interest and resources and is undergoing constant advance. The development of such a transponder tag for the use described above comes closer each year and one day should be attained.

However, there are two different marking systems in existence which can be used as the two systems 11 and 13, respectively.

A) The Magnetomechanical, Resonant Marker:

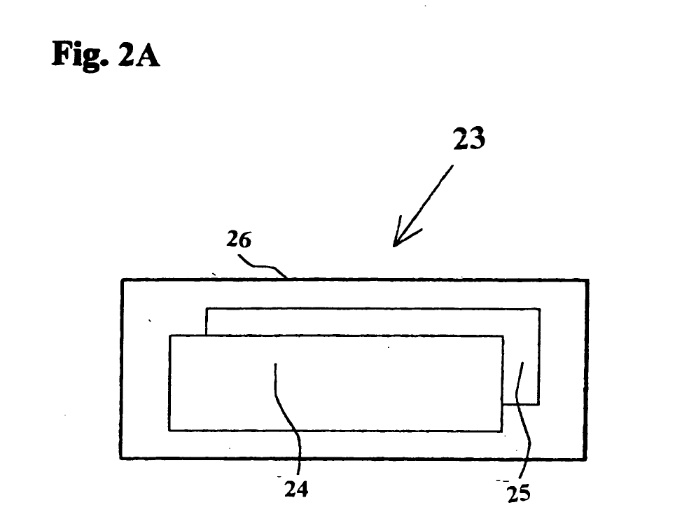

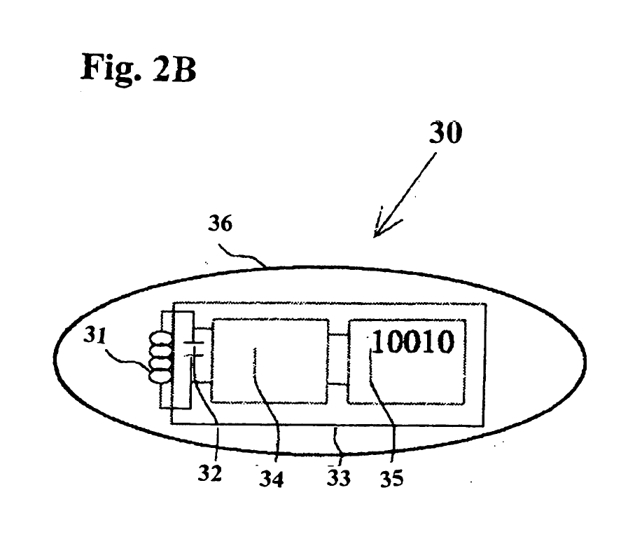

Referring now to FIG. 2A, the magnetomechanical, resonant marker 23 includes two metal strips 24, 25, a magnetomechanical strip and a biasing strip, contiguous with one another, enclosed in a water-tight casing 26. As presently constituted, the marker is about 43 mm in length, 13 mm in width, and approximately 3 mm in thickness and is enclosed in a plastic casing. However, one embodiment of the magnetomechanical marker 23 could include a housing 26 made from silicone, such as a silicone pot, in order to permit the magnetomechanical marker 23 to survive heat sterilization. Typically, magnetomechanical markers emit a signal having a frequency of between 50 kHz and 150 kHz. Advantageously, the marker 23 is highly detectable, essentially 100% out to 75 cm and very accurate. It is possible to detect the presence of one or more tags in an instant and false positive signals do not occur. It is unaffected by intervening tissue and resistant to metallic shielding. Testing of this tag has been described in an article printed in the journal Surgery in March of 2005.

Magnetomechanical resonant markers are very inexpensive and thus virtually ideal for tagging the surgical sponge, which is typically used only once then discarded. Since the surgical sponge is, by far, the implement most commonly left in the body after surgery and when retained likewise causes the most severe complications, universal use of this tag on sponges could virtually eliminate this major mishap and benefit thousands of patients.

The magnetomechanical or resonant tag, as it presently exists, however has a few limitations which make it not ideal for tagging all surgical implements—its present size makes it rather cumbersome for attachment to smaller articles such as hemostats, scalpel handles, and possibly the 4″ by 4″ gauze pads, and the present plastic enclosure is unable to survive heat sterilization, the method generally used on surgical instruments. As noted above, one proposed embodiment of magnetomechanical marker can include a silicone housing, to enable the marker to survive heat sterilization. It also has the limitation that this type of tag does not carry coded information. Detection of the magnetomechanical resonant marker only indicates whether it is present or not.

B) RFID Transponder Markers:

Referring now to FIG. 2B, there is shown one example of an RFID radiofrequency marker 30, which may be used with a multiplex detection system of the instant invention. The marker 30 includes an antenna 31 which receives a power pulse from a remote detector-interrogating antenna (for example, antennas 14 or 21 of FIG. 1) to charge a capacitor 32. This capacitor 32 becomes the power source for the operation of the unpowered radiofrequency marker, which has an integrated switch, having an integrated circuit 33 which has a reading function, a carrier frequency modulating function at 34 and a read-only memory portion 35 with a burned-in code marked here as ‘10010’. The radiofrequency integrated chip 33, together with the antenna 31, is encapsulated in enclosure 36, resistant to blood, water or saline solution.

RFID transponder markers or tags, as shown in FIG. 2B, are generally more sophisticated than the earlier magnetomechanically resonant tags, described above and shown if FIG. 2A, and therefore have certain additional advantages over mere magnetomechanical tags. RFID tags generally operate at frequencies ranging from 30 kHz to 2.45 GHz. Depending on the carrier frequency used and the type of construction, RFID tags vary significantly in cost, size, and their resistance to shielding and intervening tissue.

Further, a wide variety of RFID tags are now commercially available. A number of manufacturers produce radiofrequency (RFID) markers. Most notable of these manufacturers are Texas Instruments, Hughes Identification Devices, Destron-Fearing Corporation. Modern RFID tags can also provide significant amounts of user accessible memory, sometimes in the form of read-only memory or write-once memory. The amount of memory provided can vary, and influences the size and cost of the integrated circuit portion of an RFID tag. Typically, between 128 bits and 512 bits of total memory can be provided economically. For example, an RFID tag available from Texas Instruments of Dallas, Tex., under the designation “Tag-it” provides 256 bits of user programmable memory in addition to 128 bits of memory reserved for items such as the unique tag serial number, version and manufacturing information, and the like. Similarly, an RFID tag available from Philips Semiconductors of Eindhoven, Netherlands, under the designation “I-Code” provides 384 bits of user memory along with an additional 128 bits reserved for the aforementioned types of information.

For the application in question, the intraoperative identification of surgical implements, this discussion will divide them into two main categories, the lower frequency tags and the higher frequency tags. Each of these two main types of tag have advantages and disadvantages and the choice of which type to use will be made by the personnel of each surgical department.

1) Lower Frequency RFID Tags:

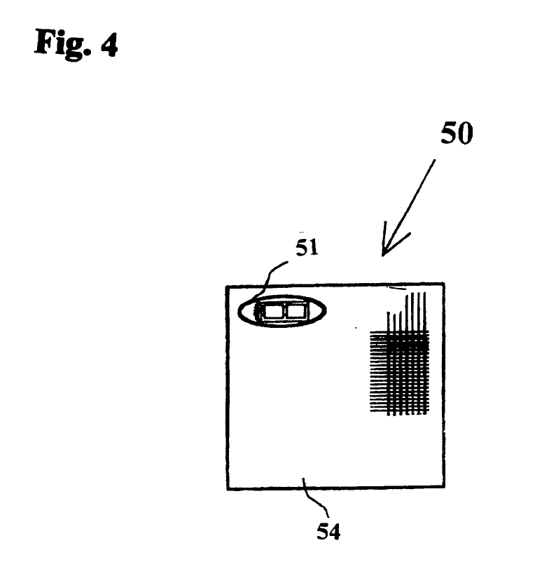

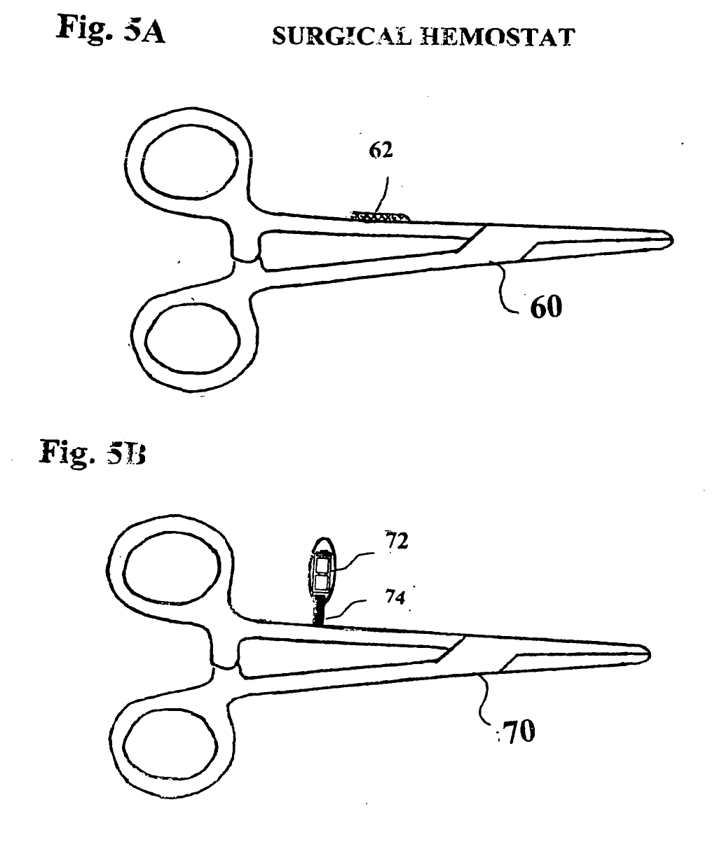

The lower frequency RFID tags generally operate in the range of from 30 kHz-500 kHz, but more preferably in the range from 100 kHz to 150 kHz. Advantageously, they can be made quite small, for example the size of a grain of rice (about 11 mm by 2.5 mm), and when enclosed in glass, polymer or a silicone pot, are robust in construction, allowing repeated sterilization by virtually any method currently in use—heat, gas, chemical, or gamma radiation. As such, lower frequency RFID tags can be attached to smaller articles such as hemostats (as shown in FIG. 5B), scalpel handles, and possibly the 4″ by 4″ gauze pads (as shown in FIG. 4). Additionally, RFID tags can carry coded data. They are relatively inexpensive to manufacture, and the lower frequency tags are more resistant to shielding than the higher frequency tags.

However, these lower frequency RFID tags, the relatively simpler members of the RFID family, have a narrow bandwidth and are therefore slower to read. Additionally, the lower frequency tags, generally, only give identifying data when a single tag is in the interrogation zone at a time. Nonetheless these lower frequency RFID tags can still carry enough coded data to give separate identity to many millions of tags, enough to identify every surgical item in a large hospital.

Absent an anti-collision mechanism in the firmware of the tag, the narrow bandwidth of the lower frequency tags prevents bulk reading of multiple tags at once, rather requiring that each tag be read individually, as noted above. This aspect may be regarded as a disadvantage in some surgical departments since, if multiple tags cannot be identified at the same time, surgical implements generally cannot be individually identified while in the patient (as there will likely be more than one tag at a time in the same interrogation zone). Nonetheless, and more importantly, scanning of the patient at the conclusion of surgery and prior to wound closure can still be used to determine whether there are any tagged items left in the wound, even though not individually identified while still in the wound. If RF evidence of any residual tagged items are found at the conclusion of surgery, the surgeon will be alerted to continue the search until all such items are removed, and any items so discovered can then be individually scanned and identified upon removal. More particularly, lower frequency RFID readers presently give only two responses—no tag found or one tag found, along with its ID data. Normally, if two or more tags are in the interrogation zone, the presently given response will be the same as “No tag found”. However, through a modification made to the lower frequency systems, the reading device will be made to give a third response which indicates the presence of multiple tags. Such modification is the subject matter of co-pending, U.S. patent application Ser. No. (Not Yet Assigned), filed on Dec. 9, 2005, and entitled SURGICAL IMPLEMENT DETECTOR.

Under this modification, these lower frequency readers will give three responses—1) no tag found, 2) one tag found (with its identification number), and 3) more than one tag found, without giving specific identification. This modification to existing technology will allow the reader to positively detect the presence of one or more tags within a wound, even though not identifying each tag specifically. This aspect can be employed at the conclusion of surgery just prior to closure of the wound to verify that no tagged items remain behind. If any tags are found at this time, any such retained items, once detected, can be removed, and then individually identified one at a time as they are recovered. Even these small, simple tags can carry a large amount of coded data, sufficient to identify each implement in use within a large hospital. If the operating room personnel decide to utilize the lower frequency system, despite the fact that its narrower bandwidth requires that each tag be read individually, they retain the advantages of smaller size, lower cost, and virtual elimination of potential shielding, while still having the ability to take an inventory of individual tagged items removed from the surgical wound.

Individual scanning of each tagged item upon removal will assure that each item is detected and read, eliminating concern that a given item may fail detection because of shielding or being out of reading range. Even when one of the lower frequency RFID tags is affixed directly to a large metal instrument such as a retractor, an individual reading can still be obtained as the scanner (or reader) is brought close to the tag. Alternatively, an anti-collision mechanism may be provided in the firmware of the RFID tags to allow a plurality of tags to communicate simultaneously.

2) Higher Frequency RFID Tags:

The higher frequency RFID tags are those that operate above the 1 MHz range. Higher frequency RFID tags exist that operate, for example, at 13.56 MHz, in the 850 to 950 MHz range, and at microwave frequencies (i.e., 2.45 to 2.55 GHz). The added bandwidth provided by these higher frequency RFID tags permits the system to reliably process multiple tags in the interrogation zone in a short period of time. This will allow a number of implements to be identified and inventoried substantially simultaneously, such as the items lying on the instrument table, or multiple implements contained within the wound cavity.

Higher frequency RFID markers tend to be significantly more sophisticated than lower frequency RFID devices. However, the higher frequency RFID tags have the limitation over those of lower frequencies, in that: they are considerably more expensive to manufacture; they tend to be relatively bulky; and their detection is more affected by intervening tissue and by shielding from adjacent metallic objects.

Based on consideration of such respective advantages and disadvantages, the ultimate choice of which type of RFID system to utilize (higher or lower frequency) in a given surgical department will vary from one department to another and be individually made by the personnel of each institution. Such a decision will balance the advantages of the small, relatively inexpensive, low frequency tags which are more resistant to shielding but require individual reading, against the larger, higher frequency tags with their ability to be read simultaneously in numbers, allowing them to be read as a group and identified while still in the wound.

Note that, although more expensive than magnetomechanically resonant tags, RFID tags are designed to withstand repeated sterilization. As such, they can be attached to metal instruments which are sterilized and reused multiple times, which reduces their average cost per use.

Metal Detection:

One possible limitation to the use of radiofrequency systems in the detection of surgical implements is that, operating in the radiofrequency spectrum, the detection of such radiofrequency tags (i.e., RFID and magnetomechanical) can be affected by the close proximity of the tag to metal, i.e., the “shielding” effect. Since RFID tags are more likely than magnetomechanical tags to be attached to metal instruments, shielding is more likely to be a problem with them. In using RFID tags, the effect of shielding can be reduced by using lower frequency tags, or by using a nonmetallic spacer to attach such higher frequency RFID tags to metallic objects. However, if it is desired to further reduce the possible effects of shielding, another possible detection system may be included in the multiplex detection system 1 of FIG. 1.

Referring back to FIG. 1, there is shown a detection system 15, which operates as a metal-detection system to detect any metal within the wound, whether a retractor, clamp, hemostat, or the like, based solely on the presence of metal and irrespective of the presence or absence of a marker or tag. The inclusion of a metal detection system 15 can be useful in circumstances wherein there exists the possibility of shielding from metal instruments used in the operation. Using a metal detection system during such an operation, in combination with one or both of the above described marker detection systems, provides a further way to prevent any metallic instruments and/or other implements from being overlooked.

Such a metal detection system 15, may work to detect metallic objects in the wound site, both ferrous and nonferrous. The metal detection system 15 utilizes conventional electronic circuitry adapted for the detection of metal objects and includes a field-generating means within the system 15 and an antenna, such as antenna 16 or 21 of FIG. 1, for generating electromagnetic radiation.

The electromagnetic radiation generated by the system 15 couples with any metallic instruments within the surgical wound, and the overall inductance of the antenna is accordingly increased. The system 15 analyzes this change in inductance of the antenna to detect the presence of metallic instruments within a surgical wound. Preferably, the system 15 uses a pulsed interrogating field. The antenna 16 or sensor coil looks for decay in the eddy current of the metallic object. Advantageously, in a preferred metal detecting system, no movement of the sensor coil would be required. One possible limitation of the use of a metal detection system is that, such a system gives no individual identifying information regarding the particular implement detected. Additionally, such a system will not, of course, detect nonmetallic implements.

X-Ray:

Another possible detection modality that can be used as one of a plurality of modalities in connection with the instant invention is that of x-ray. More particularly, for purposes of the instant invention, it is understood that metallic implements may be detected by the taking of an x-ray. Taking x-rays of surgical patients (not shown), after surgery, has been a time-honored method of detection for many years and is excellent for detecting all manner of metallic instruments. X-rays however do not reliably detect nonmetallic items, such as retained sponges, which constitute the greatest number of retained implements. Although sponges generally contain radiopaque markings, they are not infrequently overlooked or mistaken for other items present in the postoperative patient. Furthermore, x-ray detection has the disadvantages of being expensive, cumbersome, time-consuming, and involves exposing the patient (and personnel) to radiation. As a result, use of x-rays in surgical detection of implements in a wound cavity has never come into widespread usage. However, in the case where doubt remains, after using at least one other modality to locate surgical implements within the surgical wound, x-ray can be reserved as a fail-safe means to ensure that all implements visible on x-ray (i.e., metallic instruments, sponges having X-ray detectable markers, etc.) have been located.

As can be seen from the foregoing, the multiplex detection system of the embodiment of FIG. 1 can reliably detect a variety of devices in use in a surgical operation, utilizing a combination of separate detection modalities, each having complementary capabilities.

Referring back to FIG. 1, in the present embodiment, the system 11 includes an antenna 12 for detection of magnetomechanical resonant markers that may be in the surgical wound 20. The system 11 detects magnetomechanically resonant marker tags attached to objects including surgical sponges (also known as laparotomy pads), gauze pads and the like. Antenna 12 may be attached to the system 11 for remote detection or the multiplex detection system of the instant invention may further include a handheld antenna 21 that can be manipulated by a surgeon 22, in order to provide information to the system 11.

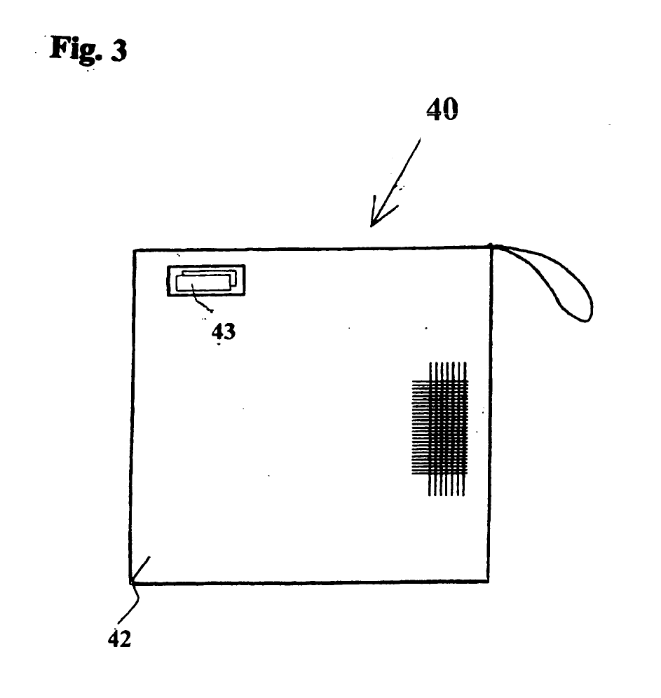

More particularly, the system 11 includes a first detection circuit, which comprises a first field generating circuit and a first antenna 12 or 21 for generating electromagnetic radiation. The electromagnetic radiation couples with markers affixed to surgical sponges/laparotomy pads (43 of FIG. 3), and gauze pads within the surgical wound and the first antenna 12 receives the response from the marker. The system 11 analyzes this response to detect the presence of a marker attached to surgical implements within a surgical wound. In the system 11, a magnetomechanical marker exhibits mechanical resonance at a resonant frequency in response to the incidence thereon of an alternating electromagnetic interrogating field, wherein the marker is provided with a signal-identifying characteristic. The resonance is preferably detected by providing the interrogating field in the form of a pulse and sensing the ring-down decay in amplitude of the electromagnetic signal transmitted by the resonating marker. The system 11 preferably operates at a frequency of 50 KHz to 150 KHz. Alternatively to, further to, and/or in accordance with the marker shown in FIG. 2A, the magnetomechanical marker may include a magnetostrictive amorphous strip, a piezoelectric crystal circuit or a tuned LCR circuit, which has a characteristic resonance frequency. One such magnetomechanically resonant marker, and a surveillance system incorporating the marker, is disclosed in U.S. Pat. No. 4,510,489.

The RFID detection system 13 of the multiplex detection system 1 detects and reads RFID tags attached to various implements. System 13 includes an antenna 14 providing marker detection functionality, using remote detection. However, as with the system 11, the system 13 can additionally include a handheld antenna 21 manipulated by a surgeon 22.

System 15 of the multiplex detection system 1 detects ferrous and non-ferrous metallic objects including surgical instruments and the like. System 15 includes an attached antenna 16 for remote detection or, as with the previous systems, be provided with a handheld antenna 21 manipulated by a surgeon 22.

Usage of Markers and Tags:

Referring to FIG. 1, the system 11 is used to detect magnetomechanically resonant marker tags typically affixed to larger, disposable non-metallic surgical items within the surgical wound. These magnetomechanically resonant markers 43 are very inexpensive in cost, reliably detectable, and particularly suited for use on disposable implements, such as surgical sponges or laparotomy pads or gauze pads. Referring more particularly to FIG. 3, there is shown at 40 attachment of magnetomechanical resonant marker 43 (which may be of the same type as marker 23 of FIG. 2A) to a surgical sponge (laparotomy pad) 42, which is typically fabricated of soft absorbent cotton cloth, generally 14 or 18 inches square. Such sponges are by far the surgical item most frequently lost track of and left behind and when so retained are also most likely to cause serious, even fatal complications. A retained sponge is termed a “gossypiboma”, roughly, a “cotton tumor” and the medical literature is replete with incidents of this mishap. The magnetomechanically resonant marker used with the system 11 of FIG. 1 is optimal for detecting these sponges. Indeed, were all such sponges to be provided with these markers, the problem of retained sponges could be nearly eliminated and thousands of patients would benefit.

The system 13 is designed to detect and read RFID transponder tags attached to metal instruments which are reused and sterilized repeatedly, such as clamps, hemostats, scalpels, and, may also attached to other smaller implements on which tracking is desired, such as 4″ by 4″ gauze pads, rubber tubing and the like. Additionally, the system 13 can be designed to detect and read individual RFID tags, which can be coded with identifying data, allowing electronic inventory of all RFID-bearing implements.

Referring now to FIG. 4, there is shown at 50 the incorporation of RFID marker 51 (which may be of the same type as marker 30 of FIG. 2B) in gauze pad 54, which is typically 4 inches square. Illustrated in FIG. 5A is a hemostat 60 provided with an RFID tag 61, directly attached to the body of the instrument. Alternatively, FIG. 5B shows a hemostat 70 provided with a RFID marker 72, attached by nonmetallic spacer 70.

More particularly, the system 13 of FIG. 1 detects and reads “smart” RF markers; or commercially available RFID targets, integrally enclosed in a glass, silicone or polymeric liquid-tight package that is resistant to washing and laundering as well as any sterilization procedures in use. In the system 13, as described in connection with FIG. 2B, the RFID marker has an antenna 31 and a memory 35 for storing a predetermined code. The RFID marker is powered by a voltage induced in the antenna by the electromagnetic interrogating field and is operative in the presence of the interrogating field to transmit the predetermined code as a change in the impedance of the antenna. One embodiment of an RFID marker detection system is disclosed by EP 0 967 927 B1 to Fabian and Anderson, incorporated herein by reference.

In an alternate embodiment, the marker may be a commercial RFID tag. Commercial RFID tags operate without need for a battery, because they include a capacitor circuit which is charged by an interrogating electromagnetic field carrier wave, which powers an integrated chip, typically including a burned-in code in a read-only memory. The capacitor power is additionally used to modulate the carrier wave to encode and broadcast the code. The modulated carrier wave is received by the interrogating antenna and is decoded to identify the code and relating it to the identity of an object using a look-up table.

As noted above, RFID tags transmit their code as a modification of a carrier frequency. Carrier frequencies in the lower frequency range vary from 30 to 500 kHz, but are most preferably between about 100 kHz to 150 kHz. Carrier frequencies in the higher frequency range is most preferably about 13.56 MHz, and in ultra high carrier frequencies are most preferably between 850 MHz to 950 MHz. Microwave frequencies between 2.4 GHz to 2.5 GHz are also used in different applications, but may cause heating of tissue and are not, necessarily, envisioned in this application. Carrier frequencies can be modulated by pulse width modulation (PWM), pulse position modulation (PPM), frequency-shift keying modulation (FSK).

As noted above, both the system 11 and the system 13 detect objects to which an electronic marker or tag has been attached. The marker is interrogated by an antenna which generally uses radiofrequency electromagnetic waves. The presence of metal, depending upon its amount and ferromagnetic properties, can attenuate the electromagnetic field of a tag in its immediate vicinity and interfere with its detection, whether the tag is magnetomechanically resonant or RFID. This phenomenon of signal attenuation by metal is referred to herein as “shielding” and its potential interference with detection by the systems 11 and 13 is a determining factor for including the metal detection system 15 in a multiplex detection system, in addition to, or in place of one of the systems 11 or 13.

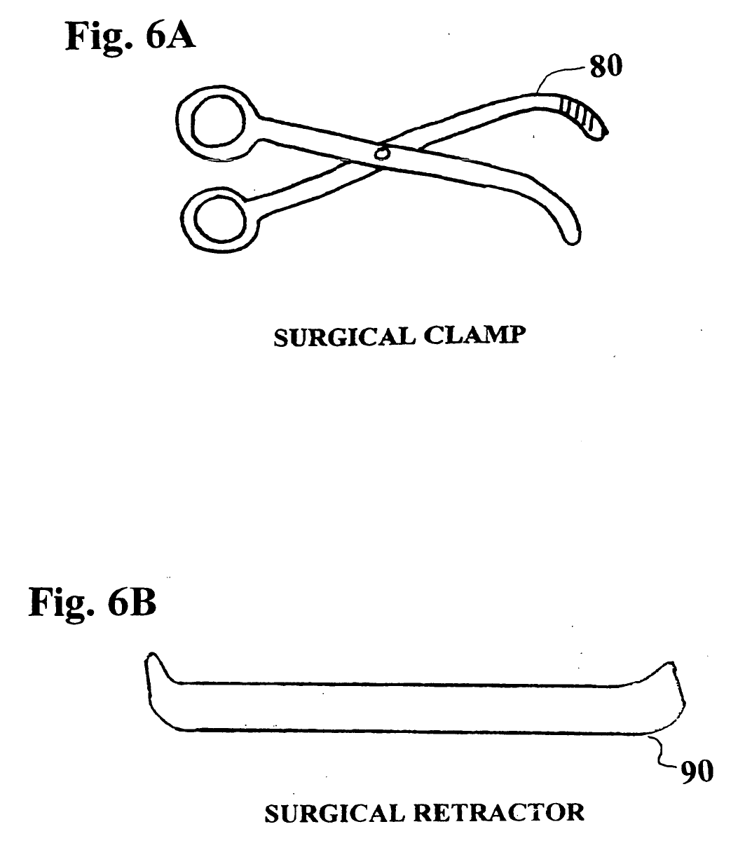

As such, the system 15 of FIG. 1 is designed to detect metallic objects solely based upon their metal content without need for an affixed marker. For example, referring to FIGS. 6A and 6B, there are shown examples of two different metallic instruments, a clamp 80 and a retractor 90, either of which would be detected by the metal-detection system 15 of the embodiment of FIG. 1. Note that, if a system such as 15 of FIG. 1 is used, the instruments 80, 90 are detected by the system 15 solely based on their metal content, irrespective of the presence of an electronic tag, and as such, no radiofrequency markers are needed for their detection, by this system.

Metal detectors use different physical principles to detect a metallic object. Typically, an AC circuit with a coil acts as a transmitting antenna. When a metallic object is brought in close proximity, eddy currents are induced in the metallic object, thereby increasing the inductance of the search coil.

The electronic circuit of the metallic object detection system 15 may detect the change in the inductance of the sensor coil; the change of phase of voltage impressed, or current passing through the search coil, or rate of change of current or voltage as a sensor coil is swept over a metallic object. In one particular embodiment, the increase in induction is detected as a change in the voltage-current characteristics, wherein the system 15 is looking for changes in the voltage-current relationships.

System 15 may more reliably detect a metallic object when the sensor coil is swept across the target area. If the sensor coil is maintained stationary, depending on the system, it may no longer observe a change of inductance in the coil. In a metal detector of the system 15 of FIG. 1 using this type of sensor coil circuit, generally some movement of the antenna with respect to the surgical cavity is required. As such, in the presently described embodiment, the antenna of the system 15 mounted upon a cart may be energized as the cart is moved into position next to the patient 19. Such movement is sufficient to establish whether a metallic instrument is left behind within a surgical cavity.

Another embodiment of the instant invention may use a handheld version of the sensor coil (21 of FIG. 1) with the system 15.

In such an embodiment, movement of the sensor coil over the surgical cavity detects any remaining metallic surgical instrument. The same antenna or sensor coil looks for decay in the eddy current of the metallic object.

In a further, preferred embodiment of the system 15, a pulsed interrogating field is used, which does not require movement of the sensor coil. In such a pulsed interrogation system, the search coil is energized by a current pulse. After the pulse is interrupted, the decaying magnetic field emanating from the coil induces eddy currents in a nearby conductive object. Those currents, in turn, produce a decaying magnetic field, which may be detected by voltage induced in a detection coil. In this way, an indication is provided that metal remains in the surgical wound 20.

The metal detection system 15 can provide further assurance that a tagged metallic item has not escaped detection because of shielding, by detecting the actual metal in the instruments, and not relying on the presence of a tag.

In one example of a test of a metal detection system for use in one embodiment of the instant invention, the ‘AUTO SCAN SECURITY DETECTOR’ metal detector system, manufactured by WHITE ELECTRONICS, INC., was used to detect metallic objects in a wound cavity within a cadaver. The AUTO SCAN SECURITY DETECTOR was positioned over the body surface and scanned back and forth. This movement enhanced the detection of metallic objects, since movement causes a change in the magnetic coupling between the detector and metallic object which change is indicated by an audible signal. Sensitivity of the detector can vary with the spatial relationship between detector and the metallic object, and can be somewhat reduced if the metallic object is oriented perpendicularly to the detector. However, since the surgical cavity is generally flat and has a limited depth, retained surgical instruments tend to lie flat in the cavity and were easily, quickly, and reliably detected by this device. Note that, the specific techniques, conditions, materials, proportions and reported data set forth in the above example to illustrate the principles and practice of the invention are exemplary and should not be construed as limiting the scope of the invention.

In Operation:

Generally stated, the invention employs a combination of discrete systems to identify surgical implements within a surgical wound. As stated above, two or more of the modes may be chosen to work together to electronically “inspect” or “interrogate” the surgical wound, in complementary fashion, to detect surgical implements. For example, if the two radio marker systems are chosen, they may work in complementary form to use the inexpensive magnetomechanical markers on sponges, while using the more expensive RFID markers on smaller implements, such as gauze pads or hemostats, not suited for larger magnetomechanical markers. In this way the systems can be chosen to cost effectively provide detection of all devices. Similarly, one of the two radio marker types may be provided on all non-metallic implements, while the metallic include no marker, such that the non-metallic devices are detected by the chosen one of systems 11 or 13, while the metallic devices are detected by the metal detection system 15. As can be seen, the multiplex detection system can be optimized using at least two of the disclosed detection modalities, and possibly three, if desired.

Consequently, in combination, any two or more of the described modalities may be included in the present multiplex detection system to vastly reduce the possibility that surgical implements, non-metallic or metallic, will be left behind within a surgical cavity.

Further, depending on shielding and/or other radio-electronic interference in the operating room, the chosen modes of operation of the systems 11, 13 and 15 can operate sequentially or simultaneously. However, since system 11, system 13, and system 15 all use electromagnetic waves to interrogate the surgical wound for the presence of electronically tagged items and metallic objects prior to wound closure, there exists the potential for electromagnetic interaction between the three systems. Preferably, the different modalities can be chosen to operate in any order. Additionally, such interference can be minimized by operating the systems 11, 13 and 15 at different electromagnetic radiation frequencies, thereby preventing deleterious interference between the systems in detecting implements present in the surgical wound.

For example, since it has been discussed that shielding can be reduced by taking certain measures in an RFID marker system, such as distancing the tag from the metallic instrument using a non-metallic spacer, in a system using both RFID and metal detection, it is not necessary that the metal detection modality be used in advance of RFID detection. In systems using all three detection modalities, magnetomechanical, RFID and metal detection, such modalities can be operated as desired. For example, although objects bearing marker tags are generally detected either by the magnetomechanically resonant or by the RFID detection systems, the presence of metal close to the tags may cause local attenuation of the radiofrequency field resulting in “shielding”, which can diminish the efficiency of detection. In such a case, metal detection system 15 may be employed to specifically detect any metallic implement present in the surgical cavity. In one particular preferred embodiment, the three systems interrogate the surgical wound during alternating time periods so that they each detect their appointed implements independently of one another.

Similarly, in a system using only the two marker systems, there is no preference as to the order in which the detection systems are operated.

Further, as shown in FIG. 1, the systems of the selected detection detectors (i.e., systems 11, 13 and 15 for the embodiment shown in FIG. 1) may be conveniently mounted on a rollaway cart 17, with antennae that are fixed to the detection devices and/or attached to a handheld sensor coil/antenna that is scanned or moved over the surgical cavity.

If desired, any or all three systems 11, 13 and 15 can be powered by a battery, preferably a rechargeable battery, or AC power. Further, if desired, any or all three systems can be mounted upon a rollaway cart, and/or may include handheld antennae.

The multiplex detection system 1 of FIG. 1 does not use any X-ray equipment or require radiation protection and thus is lightweight and highly portable, allowing it to be easily brought into an operating room, and into close proximity to the patient, when needed.

Significant advantages are realized by practice of the present invention.

Having thus described the invention in rather full detail, it will be understood that such detail need not be strictly adhered to, but that additional changes and modifications may suggest themselves to one skilled in the art, all falling within the scope of the invention as defined by the subjoined claims. Under certain circumstances the combination of systems may include more than three systems detailed above, or as few as two systems.

I claim:

1. A multiplex system for detecting surgical implements within a surgical cavity of a patient, comprising:
a first system for electronically detecting surgical implements;
a second system for electronically detecting surgical implements, said second system being different from said first system; and
both said first system and said second system being used to inspect the surgical cavity for surgical implements.

2. The system of claim 1, wherein said first system is chosen from the group including, magnetomechanically resonant marker tags, RFID markers, a system designed to detect metallic objects based upon their metal content.

3. The system of claim 2, wherein said second system is additionally chosen from the group including, magnetomechanically resonant marker tags, RFID markers, a system designed to detect metallic objects based upon their metal content

4. The system of claim 2, wherein said system designed to detect metallic objects based upon their metal content includes a metal detector and sensing coil.

5. The system of claim 1, wherein said first system and said second system are used simultaneously.

6. The system of claim 1, wherein the use of said first system and said second system are used alternately.

7. The system of claim 1, at least a portion of said first system and at least a portion of said second system being incorporated onto a cart.

8. The system of claim 1, further including a third system for detecting surgical implements, said third system being different from said first and second systems, said third system additionally being used to inspect the surgical cavity for surgical implements.

9. The system of claim 8, wherein said third system is chosen from the group including, magnetomechanically resonant marker tags, RFID markers, a system designed to detect metallic objects based upon their metal content.

10. The system of claim 1, wherein said first system detects a surgical implement by detecting a radiofrequency marker associated with said surgical implement.

11. The system of claim 10, wherein said radiofrequency marker operates at a frequency of between 50 kHz and 500 kHz.

12. The system of claim 10, wherein said radiofrequency marker operates at a frequency of between 50 kHz and 150 kHz.

13. The system of claim 12, wherein said radiofrequency marker is a magnetomechanical marker.

14. The system of claim 12, wherein said radiofrequency marker is an RFID marker.

15. The system of claim 11, wherein said second system detects a surgical implement based upon the metal content of said surgical implement.

16. The system of claim 15, wherein said second system includes a metal detector and sensing coil.

17. The system of claim 15, wherein said first system and said second system are used simultaneously.

18. The system of claim 15, wherein the use of said first system and said second system are alternated.

19. The system of claim 11, wherein said second system detects a surgical implement by detecting a second radiofrequency marker associated with said surgical implement.

20. The system of claim 19, wherein one of said radiofrequency marker and said second radiofrequency marker is a magnetomechanical marker, and wherein the other of said marker and said radiofrequency marker is an RFID marker.

21. The system of claim 20, wherein said first system and said second system are used simultaneously.

22. The system of claim 20, wherein the use of said first system and said second system are alternated.

23. A method for detecting surgical implements within a surgical cavity of a patient, comprising:
providing a first system for electronically detecting surgical implements;
providing a second system for electronically detecting surgical implements, the second system being different from the first system; and
utilizing both the first system and the second system to inspect the surgical cavity for surgical implements.

24. The method of claim 23, wherein the first system is chosen from the group including, magnetomechanically resonant marker tags, RFID markers, a system designed to detect metallic objects based upon their metal content.

25. The system of claim 24, wherein the second system is additionally chosen from the group including, magnetomechanically resonant marker tags, RFID markers, a system designed to detect metallic objects based upon their metal content.

26. The system of claim 24, wherein the system designed to detect metallic objects based upon their metal content includes a metal detector and sensing coil.

27. The system of claim 23, wherein the first system and the second system are used simultaneously.

28. The system of claim 23, wherein the use of the first system and the second system are alternated.

29. The system of claim 23, at least a portion of the first system and at least a portion of the second system being incorporated into a portable unit.

30. The system of claim 23, further including a third system for electronically detecting surgical implements, the third system being different from the first and second systems, the third system additionally being used to inspect the surgical cavity for surgical implements.

31. A multiplex system for detecting surgical implements within a surgical cavity of a patient, comprising:
a first system for electronically detecting surgical implements, said first system detecting a surgical implement by detecting a first radiofrequency marker associated with said surgical implement;
a second system for electronically detecting surgical implements, said second system either detecting a second radiofrequency marker of a different type than said first radiofrequency marker associated with said surgical implement or detecting metallic objects based upon their metal content; and
both said first system and said second system being used to inspect the surgical cavity for surgical implements.

32. The system of claim 31, wherein said first radiofrequency marker is an RFID marker that broadcasts a code and said second system detects a second radiofrequency marker, said second radiofrequency marker being a magnetomechanical marker.

33. The system of claim 31, wherein said first radiofrequency marker is an RFID marker that broadcasts a code and said second system detects metallic objects based upon their metal content.

34. The system of claim 33, wherein said second system includes a metal detector and sensing coil.

35. The system of claim 31, wherein said first radiofrequency marker is a magnetomechanical marker and said second system detects metallic objects based upon their metal content.

36. The system of claim 33, further including a third system for electronically detecting surgical implements, said third system including a second radiofrequency marker, said second radiofrequency marker being a magnetomechanical marker.

37. The system of claim 31, wherein at least a portion of said first system and at least a portion of said second system being incorporated into a portable unit.

38. The system of claim 37, wherein said portable unit is a rolling cart.
